(19) United States
(12) Patent Application Publication
(10) Pub. No.: US 2023/0341379 A1
(43) Pub. Date: Oct. 26, 2023

(54) High Throughput Multiparametric Immune Cell Engager Screening Assay

(71) Applicant: Genentech Inc., South San Francisco, CA (US)

(72) Inventors: Katherine Kozak, Half Moon Bay, CA (US); Josefa dela Cruz Chuh, Santa Clara, CA (US)

(73) Assignee: Genentech Inc., South San Francisco, CA (US)

(21) Appl. No.: 18/188,665

(22) Filed: Mar. 23, 2023

Related U.S. Application Data
(63) Continuation of application No. PCT/US2021/051907, filed on Sep. 24, 2021.
(60) Provisional application No. 63/083,969, filed on Sep. 27, 2020.

(51) Int. Cl. G01N 33/50; G01N 33/58

(52) U.S. Cl. CPC G01N 33/505; G01N 33/5011; G01N 33/5038; G01N 33/582

(57) ABSTRACT

The present disclosure provides methods and systems for high throughput assays for testing immune cell engager molecules and potential immune cell engager molecules. In some embodiments, multiple parameters, for example, in connection with engagement of tumor cells by immune cells such as T cells and in connection with tumor cell death, may be analyzed from the same samples in the assays, and, in some cases, may be analyzed simultaneously. In some embodiments, the methods and systems allow for determining the kinetics of various parameters.

### CROSS-REFERENCE TO RELATED APPLICATIONS

This application claims the benefit of priority to U.S. Provisional Patent Application No. 63/083,969, filed Sep. 27, 2020, the contents of which are incorporated herein by reference in their entirety.

### FIELD

The present application relates to methods and systems for high throughput assays for testing immune cell engager molecules and potential immune cell engager molecules. In some embodiments, multiple parameters, for example, in connection with engagement of tumor cells by immune cells such as T cells and in connection with tumor cell death, may be analyzed from the same samples in the assays, and, in some cases, may be analyzed simultaneously. In some embodiments, the methods and systems allow for determining the kinetics of various parameters.

### BACKGROUND

Immune cell engagers include molecules that may bring immune cells into proximity with cells to be targeted for destruction, for example, by binding to cell surface molecules on each type of cells and serving as a bridge to bring the two cell types together. Binding of the engager to both immune cells and target cells may create an artificial immune synapse. This process may operate independently of the normal major histocompatibility complex (MHC)-dependent mechanism by which immune cells identify and kill their target cells.

A variety of assays are available to assess the activity of potential immune cell engager molecules. For example, cell death may be measured by the use of certain nuclear fluorescent stains or loss of ATP activity measured in luminescence assays, apoptosis may be measured by the use of stains whose signal depends on presence of apoptosis factors such as caspases. Related changes in a system, such as concentration of various proteins such as cytokines may also be measured. However, these assays rely on a diverse set of labeling and analysis methods.

### SUMMARY

The present disclosure encompasses methods and systems for assaying molecules that may act as immune cell engagers, for example, to determine their impact on multiple parameters connected to engagement of immune and tumor cells and tumor cell death. Methods of the present disclosure can be performed on small volumes of materials, can track various parameters kinetically, can perform different analyses of different parameters kinetically on one small volume sample, such as a well from a multi-well plate, and can allow for running many hundreds of samples in parallel. Thus, they allow for high-throughput analysis of many immune cell engager molecules in a variety of cell culture systems or types.

Exemplary methods of the disclosure include, for example:

A method of assaying the activity of a potential immune cell engager, comprising co-culturing immune cells (e.g. T cells or NK cells or PBMCs) with target cells (such as tumor or primary cells) in the presence of at least one potential immune cell engager, and assaying at least one of the following parameters: (i) death of target cells, for example based on loss of nuclear stain, (ii) apoptosis of target cells, for example based on caspase 3/7-dependent labeling of cells, (iii) change in ATP concentration (e.g. in a Cell Titer Glo® assay, which uses a luminescent label); and (iv) change in concentration of at least one analyte from the co-culture supernatant, such as a cytokine, T cell activation factor, or chemokine. In some embodiments, the cells may be co-cultured for example on a multiple-well plate (e.g. a 96 or 384 well plate), optionally wherein the target cells, such as tumor or primary cells, are stained with a dye, for example where cells are stably transfected with a nuclear fluorescence protein, for example, using a lentivirus vector, or using another transfection system. In some embodiments, target cells are first cultured and then immune cells are added. In some embodiments, immune cells are first cultured and then target cells are added. In some embodiments, the target cells and immune cells are incubated with at least one potential immune cell engager for a period of time, such as for at least 18 hours, or at least 24 hours. In some embodiments, certain parameters, for instance, may be assayed after 2, 4, 6, 12, 18, 24, 36, 48, 72, 96 or more hours. In some embodiments, one or more of assays (i) to (iv) is performed more than once, such as, for example, every 2, 4, or 6 hours. In other cases, certain parameters may be monitored continuously, such as using an automated imaging apparatus such as a cell plate imager. In some embodiments, kinetics of cell death, apoptosis activity, and/or ATP and cytokine concentration changes may be determined.

In some embodiments, cells are co-cultured in wells of a cell plate or similar structure, for example, so that they may be monitored in a plate imaging apparatus. In some cases, tumor cells are plated at, for example 1000-50,000 cells/well 5000-30,000 cells/well, such as at 5000-20,000 cells/well, or 8000-15,000 cells/well, or 8000-12,000 cells/well, or 5000 cells/well, 7000 cells/well, 8000 cells/well, 9000 cells/well, 10,000 cells/well, 11,000 cells/well, 12,000 cells/well, or 15,000 cells/well. In some embodiments, cells are plated at, for example, 5-50 μL/well, 10-50 μL/well 20-50 μL/well, 20-40 μL/well, 20-30 μL/well, 30-50 μL/well, 30-40 μL/well, 25-35 μL/well, 10 μL/well, 20 μL/well, 25 μL/well, 30 μL/well, 35 μL/well, or 40 μL/well. For example, in some embodiments, immune cells can be plated at, for example 1000-50,000 cells/well, such as at 10,000-40,000 cells/well, 10,000-30,000 cells/well, 5000-20,000 cells/well, or 8000-15,000 cells/well, or 8000-12,000 cells/well, or 5000 cells/well, 7000 cells/well, 8000 cells/well, 9000 cells/well, 10,000 cells/well, 11,000 cells/well, 12,000 cells/well, or 15,000 cells/well. In some embodiments, immune cells are added at, for example, 5-50 μL/well, 10-50 μL/well, 20-50 μL/well, 20-40 μL/well, 20-30 μL/well, 30-50 μL/well, 30-40 μL/well, 25-35 μL/well, 10 μL/well, 20 μL/well, 25 μL/well, 30 μL/well, 35 μL/well, or 40 μL/well. In some cases, the number of the target cells, such as tumor or primary cells, and/or the number of immune cells per well or sample may vary. Cell numbers and volumes may vary, for example, depending on the growth rate of the cells.

In some cases, the target cells are tumor cells. Tumor cells may be derived from tumor cell lines. In other cases, they may be from a donor. Tumor cells may be from any of a variety of human or mammalian cancers. In some cases, target cells are primary cells. Immune cells, in some cases, are T cells (e.g. pan T cells, CD8+ T cells, CD4+ T cells). In other cases, immune cells are PBMC cells. In other cases, immune cells can be a mixture of T cells and other types of cells such as B cells and/or NK cells. In some cases, immune cells may be NK cells.

The present invention encompasses methods of conducting multiple analyses of potential immune cell engagers, in some embodiments, from one plate of cells, and, in some embodiments, using relatively low volumes of materials and, in some embodiments, with most or all steps automated. For example, in some cases, hundreds of screens may be conducted, for example in several plates, over a short period of time, allowing for kinetic assays of multiple different parameters such as parameters associated with tumor cell death and apoptosis and changes in cytokine concentrations.

In some cases a potential immune cell engager is a molecule whose ability to engage immune cells and tumor cells is unknown and is to be assessed. In other cases, the molecule is a known immune cell engager, and the assay system is used, for example, to determine if the molecule is responsive to a particular type of tumor cell, or to immune cells from one or more specific donors.

In some embodiments, potential immune cell engagers to be assayed include antibodies, such as multispecific or bispecific antibodies that bind to targets on immune cells, such as T cells, and to targets on target cells such as tumor cells. For example, in some embodiments, antibodies are potential T cell dependent bispecifics (TDBs), which may bind to a target on T cells (e.g., CD3) and also to a target on tumor cells (e.g., a target expressed on tumor cells). In some embodiments, antibodies are potential costimulatory receptor bispecific (CRB) antibodies, which may bind to a costimulatory target on T cells, such as CD28 or ICOS, and to a target on tumor cells. In some embodiments, potential TDBs or potential CRBs may be assayed. In some embodiments, the assays herein may assess a combination of potential TDBs and CRBs. In some embodiments, the potential immune cell engagers that bind to targets on tumor and immune cells may comprise non-antibodies or may be conjugates of antibodies with other molecules. Further example targets of immune cell engagers on immune cells and on tumor cells are provided below

In some embodiments, the methods herein may be used to determine if a molecule acts as an immune cell engager; thus, the molecule tested may be a potential immune cell engager. In some embodiments, the methods herein may be conducted with a known immune cell engager, but may be conducted to determine its potency, or to determine how it acts in the presence of particular immune or tumor cells, or to determine its kinetics, or to determine its potency, or more than one of these factors. In some cases, methods herein may be conducted to determine how an immune cell engager interacts with immune cells of different types, or with immune cells from different individual donors. In some cases, methods herein may be used to compare different potential immune cell engagers or combinations of immune cell engagers.

The present disclosure also involves systems for performing the above methods. In some embodiments, systems comprise one or more of an automated cell plating device, an acoustic-controlled liquid dispenser, e.g. for adding immune cells and or a potential immune cell engager to the wells, a cell plate imaging device for monitoring fluorescent label on the cells, and an array or beads for determining cytokine concentrations in the wells.

Both the foregoing general description and the following detailed description are exemplary and explanatory only and are not restrictive of the claims. The accompanying drawings, which are incorporated in and constitute a part of this specification, illustrate certain example embodiments and together with the description, may serve to explain certain principles described herein.

### BRIEF DESCRIPTION OF THE DRAWINGS

FIG. 1 shows an example workflow for an exemplary high throughput, multiparametric, automated system for conducting assays herein.

FIG. 2 shows an exemplary optimization of cell density used for plating cells.

FIG. 3A-3E show changes in fluorescence of tumor cells over time following addition of immune cells and a T cell dependent bispecific antibody (TDB), reflecting killing and apoptosis of tumor cells. FIG. 3A shows the level of nuclear fluorescence intensity (NucLight™) after 1 or 3 days after addition of immune cells and TDB. FIG. 3B shows co-cultured tumor cells as a 3D spheroid (white) and Cytolight™ stained immune cells (dark grey) incubated over a period of time (left to right panels) with a TBD (top row) or without a TBD (bottom row). FIG. 3C shows single cell killing activity in one 86,400 sub-well of a micro-well 384-well plate, showing individual tumor cell staining and caspase 3/7 fluorescent labeled spots. FIG. 3D shows changes in intensity of nuclear fluorescent dye and caspase 3/7-dependent fluorescent dye in tumor cells in the presence of immune cells and an immune cell engager as target cell counts (FIG. 3D) and as percent target cell killing (normalized) (FIG. 3E).

FIG. 4A-4F provide data showing other parameters associated with engagement and killing of tumor cells, such as endpoint killing and changes in cytokine concentrations. FIG. 4A shows tumor cell killing based on metabolism (ATP) readout across 4 different cell lines (BT474, NCIH292, COV413B, and COV362) with 2 different TDBs (NLR 4D5 and NLR 2C4). FIGS. 4B, 4C, and 4D show changes in concentrations of IL-6, IFNg, and IL-2, respectively in supernatants taken from wells (upper curves), in comparison to a non-tumor target control TDB (bottom curves) FIGS. 4E and 4F show MFI signal of 2 analytes (IL-6 and IL-2) treated with 60 nM TDB over time with 4 different TBDs.

FIG. 5 shows a determination of the percentage of CD8+CD69+ T cells by flow cytometry of immune cells isolated from 384-wells.

FIG. 6A-6B show differences in cell populations upon incubation with TDB and CRB. FIG. 6A shows differences in concentrations of granzyme B, IL-10, MIP1b, IFNγ, IL-2, TNFα at high (dark circles) and low (light circles) doses of TDB in a co-cultured cell supernatant, with TDB alone or a combination of TDB and CRB. FIG. 6B shows, in the left panel, differences in the percentage of CD8+ and costimulatory receptor+(CoStim+) T cells after incubation with no TDB and with a TDB after 1 and 3 days, and in the right panel, the percent CD8+CD25+ T cells (Teff) with or without TDB after 1 and 3 days.

FIG. 7A-7F show affinity and kinetic data. FIG. 7A shows a schematic of different TDBs binding to Her2 in either a proximal (p) or distal (d) fashion, and binding to CD3 with either high (hi) or low (10) affinity. FIG. 7B provides the relative affinities of the individual anti-Her2 or anti-CD3 arms of the TDBs. FIG. 7C provides kinetic traces for the 2 TDBs, showing greater loss of cells for the higher affinity TDB treatment. FIG. 7D shows the conversion of the kinetic traces into a dose response curve for the 2 TDBs. FIG. 7E shows calculation of time it takes to kill 50% of the tumor cells for the 2 TDBs, indicating they have different rates of killing. FIG. 7F shows the dose response curves generated from the % cytolysis traces in 7E.

FIG. 8A-8G show data related to cell killing activity. FIG. 8A shows titration of a CRB co-dosed with a fixed amount of TDB. Darker curves represent higher relative concentrations of CRB to TDB. FIG. 8B shows calculation of the KT50 rates for the different treatment concentrations. FIG. 8C shows DRC calculation from the individual traces. FIG. 8D shows the percentage of target cell killing (normalized) for a titration of the CRB into 3 fixed concentrations of TDB. FIG. 8E shows the percentage of target cell killing (normalized) for titration of TDB into 4 concentrations of fixed CRB. FIG. 8F shows correlation between the maximum percentage tumor cell killing activity in a Nuclight™ red assay compared to the maximum percentage activity in a caspase 3/7 assay as described in the Example. FIG. 8G shows correlation between the maximum percentage activity in the Nuclight™ red assay compared to the maximum percentage activity in a Cell Titer Glo® assay.

FIG. 9A-9D show changes in certain analyte concentrations (FIG. 9A— IFNγ; FIG. 9B—granzyme B; FIG. 9C— IL2; and FIG. 9D— IL6) in the supernatants from wells 6, 24, and 72 hours after addition of TDB. The individual curves in each graph represent data with different TDB clones.

FIG. 10 shows a heat map ranking various CRB clones and controls based on multiple data readouts, for example KT50 of cell killing and changes in various cytokine concentrations. The cytokines were analyzed after 72 hours incubation with CD8+ T cells.

FIG. 11A-11D show increases in T cell subpopulations in immune cells from four donors over time after 1 or 3 days incubation with target cells, and with or without TDB. FIG. 11A CD8+ T cells; FIG. 11B T effector cells (Teff); FIG. 11C memory T cells (Tcm); and FIG. 11D ratio of effector to memory cells (Teff/Tcm).

FIG. 12A-12E provide further data on two donors (donors 1 and 3) from FIG. 11. FIG. 12A shows difference between donors 1 and 3 on CD8+ T cell proliferation with and without added TDB. FIG. 12B and FIG. 12C show comparisons of the rate of cell killing with increases in concentration of CRB for the two donors, and FIG. 12D and FIG. 12E show dose response curves corresponding to the data in FIGS. 12B and C.

FIG. 13A-13F show correlations of multiple readouts. FIG. 13A compares the EC50 of two different CRB molecules in the presence of target cells and either CD8+ T cells or PBMCs. FIG. 13B shows KT50 vs Max % activity of several different CRB clones in the presence of target cells and CD8+ T cells. FIG. 13C shows granzyme B vs Max % activity of various CRB clones with CD8+ T cells. FIG. 13D compares EC50 for the Her2d TDB and Her2p TDB (see FIG. 7) in the presence of CRB and with CD8+ T cells or PBMCs. FIG. 13E shows comparison between max % activity of CD8+ T cells vs Pan T cells in the presence of several CRB clones. FIG. 13F compares IFNγ vs max activity of CD8+ T cells in the presence of several CRB clones.

FIG. 14A-14C show a t-distributed stochastic neighbor embedding (t-SNE) machine learning algorithm cluster analysis of various TDB clones based on their killing and cytokine profiles over time (FIG. 14A 6 hr, FIG. 14B 24 hr, and FIG. 14C 72 hrs) to identify unique TDBs.

### DETAILED DESCRIPTION OF CERTAIN EMBODIMENTS

### Definitions

Unless otherwise defined, scientific and technical terms used in connection with the present invention shall have the meanings that are commonly understood by those of ordinary skill in the art. Further, unless otherwise required by context, singular terms shall include pluralities and plural terms shall include the singular.

In this application, the use of “or” means “and/or” unless stated otherwise. In the context of a multiple dependent claim, the use of “or” refers back to more than one preceding independent or dependent claim in the alternative only. Also, terms such as “element” or “component” encompass both elements and components comprising one unit and elements and components that comprise more than one subunit unless specifically stated otherwise.

As described herein, any concentration range, percentage range, ratio range or integer range is to be understood to include the value of any integer within the recited range and, when appropriate, fractions thereof (such as one tenth and one hundredth of an integer), unless otherwise indicated.

Units, prefixes, and symbols are denoted in their Systéme International de Unites (SI) accepted form. Numeric ranges are inclusive of the numbers defining the range. The headings provided herein are not limitations of the various aspects of the disclosure, which can be had by reference to the specification as a whole. Accordingly, the terms defined immediately below are more fully defined by reference to the specification in its entirety.

As utilized in accordance with the present disclosure, the following terms, unless otherwise indicated, shall be understood to have the following meanings:

A “plate” or “cell plate” for culturing cells means any type of structure that allows incubation of cells and observation of labels on the cells, such as fluorescent dyes. In general, a cell plate contains one or more “wells,” sometimes as many as 96 or 384 wells, which are areas on the plate where specific concentrations of reagents can be maintained so that they do not mix with the contents of other wells. Wells can be of any suitable structure for this purpose.

An “immune cell engager” refers to a molecule that is capable of enhancing the interaction of an immune cell and a target cell, such as a tumor cell or a primary cell, for example, such that the immune cell may provoke cell death or apoptosis of the target cell. In some cases, immune cell engagers bind to a target molecule on an immune cell and also bind to a target molecule on a target cell. An immune cell engager may act alone, or may act through one or more costimulatory molecules such as certain cell surface receptors. In some cases, immune cell engagers are proteins, for example antibodies. In some cases, they are bi-specific molecules, such as bi-specific antibodies, such as those recognizing a target molecule on the surface of an immune cell and another target molecule on the surface of a target cell, such as a tumor cell. A “target molecule” as used herein refers to a protein or other molecule on the surface of a cell to which an immune cell engager is intended to bind, e.g., a cell surface receptor.

A “potential immune cell engager” comprises both immune cell engagers as well as molecules being tested in the assays herein to determine whether they are immune cell engagers.

The term “antibody” herein is used in the broadest sense and encompasses various antibody structures, including but not limited to monoclonal antibodies, polyclonal antibodies, multispecific antibodies (e.g., bispecific antibodies), and antibody fragments so long as they exhibit the desired antigen-binding activity. Antibodies herein also include, for example, antigen binding fragments comprising an antigen-binding portion of a full length antibody, such as a set of heavy and light chain complementary dependent regions (CDRs) and surrounding framework regions, or heavy and light chain variable regions. Example antigen-binding fragments include Fab′, F(ab′)2, Fv, scFv, and related fragments.

As used herein, a “bispecific” immune cell engager, such as a bispecific antibody, is capable of binding to at least two different antigens or target molecules. Bispecific antibodies may have any appropriate structural format. Examples of known bispecific antibody formats include, for example, diabodies, CrossMabs, triomabs, DVD-IgGs, 2 in 1-IgG, ortho-Fab IgG, IgG-scFvs, scFV2-Fc, DART, DART-Fc, bi-nanobodies, TBTI, scFv-Fc, TandAb, orthoFab-IgG, DNL-Fab3 and others. (See, e.g., R. E. Kontermann & U. Brinkmann, Bispecific Antibodies, Drug Disc. Today, 20(7): 838-847 (2015).) Bispecific antibodies that bind to at least one target molecule on T cells, for example, and that may also act as immune cell engagers, or act to enhance immune cell engagement, include TDBs and CRBs, for example.

“T cell dependent bispecific” antibodies (TDBs) are immune cell engagers that may cause interaction of immune cells and tumor cells by binding to cellular surface targets on each type of cell. In some cases, TDBs, by bringing the two cell types together, may allow for activation of T cells, for example, without costimulation and independent of major histocompatibility complex (WIC) involvement, thus bypassing the normal two-step T cell activation mechanism.

“Costimulatory receptor bispecific” antibodies (CRBs) may bind to costimulatory targets on immune cells like T cells, for example CD28 or ICOS, and also bind to target molecules on the surface of tumor cells. In some embodiments, CRBs may enhance and extend TDB functionality. In some embodiments, potential TDBs and CRBs may be assayed singly or together.

As used herein, the term “cell” is used in the broadest sense and includes eukaryotic cells, plant cells, animal cells, such as mammalian cells, reptilian cells, avian cells, fish cells, or the like, prokaryotic cells, bacterial cells, fungal cells, protozoan cells, or the like, cells dissociated from a tissue, such as muscle, cartilage, fat, skin, liver, lung, neural tissue, and the like, immune cells, such as T cells, B cells, natural killer cells, macrophages, and the like, embryos (e.g., zygotes), oocytes, ova, sperm cells, hybridomas, cultured cells, cells from a cell line, cancer cells, infected cells, transfected and/or transformed cells, reporter cells, and the like. A mammalian cell can be, for example, from a human, a mouse, a rat, a horse, a goat, a sheep, a cow, a primate, or the like. As used herein, “immune cells” include, for example T cells, B cells, NK cells, macrophages, and monocytes in both their mature and immature forms. As used herein, “tumor cells” include cells obtained for example, from a tumor biopsy of an individual, as well as cells from cultured cancerous cell lines, and may be from any type of cancer. As used herein, a “target cell” refers to a cell that may be targeted for destruction by an immune cell, such as via apoptosis or other means. In some cases, a target cell may be killed or induced into apoptosis upon or after being bound or recognized by an immune cell. A target cell, in some embodiments, may be a tumor cell, may be a primary cell, and/or may be a cell derived from an immortalized cell line.

When plating cells herein, cells are plated “uniformly” unless otherwise specified. A “uniform” plating of cells means that the cells are plated so that substantially the same number of cells and the same volume is found in each well of the cell plate, with minimal clumping to readily allow different wells to be compared.

A sample of, for example, cells, primary cells, tumor cells, or immune cells may be obtained from an individual or subject, i.e. a donor, in some embodiments. In some embodiments, the donor is a human. However, in some embodiments, the donor may also be another mammal, such as a domestic or livestock species, e.g., dog, cat, rabbit, horse, pig, cow, goat, sheep, etc., or a laboratory animal, such as a mouse or rat.

In some embodiments, tumor cells may be derived from a particular “cancer” or suspected “cancer.” Cancers herein may include, for example, solid tumors, which comprise tumors originating from tissue cells of the body. In some embodiments, the cancer may be, for instance, breast cancer, lung cancer (including small cell lung cancer or non-small cell lung cancer, adenocarcinoma of the lung, and squamous carcinoma of the lung), prostate cancer, testicular cancer, penile cancer, esophageal cancer, tumors of the biliary tract, brain cancer (including glioblastoma), colorectal cancer, colon cancer, rectal cancer, kidney cancer (including renal cell carcinoma), liver cancer (hepatoma), adrenal cancer, cervical cancer, uterine cancer, endometrial cancer, vulval cancer, salivary gland carcinoma, squamous cell cancer of the head and neck, leukemia, lymphoma, lymphoid cancer, ovarian cancer, pancreatic cancer, bladder cancer, skin cancer such as melanoma, or urinary tract cancer.

### Exemplary Methods

Exemplary methods herein include, for example, methods of assaying the activity of a potential immune cell engager, comprising: (a) co-culturing target cells and immune cells in the presence of at least one potential immune cell engager, and (b) assaying at least one of the following parameters: (i) death of target cells, (ii) apoptosis of target cells, (iii) change in ATP concentration; and (iv) change in concentration of at least one analyte in supernatant from the co-cultured cells. In some cases, each parameter chosen for analysis is assayed within the same co-cultured cell sample.

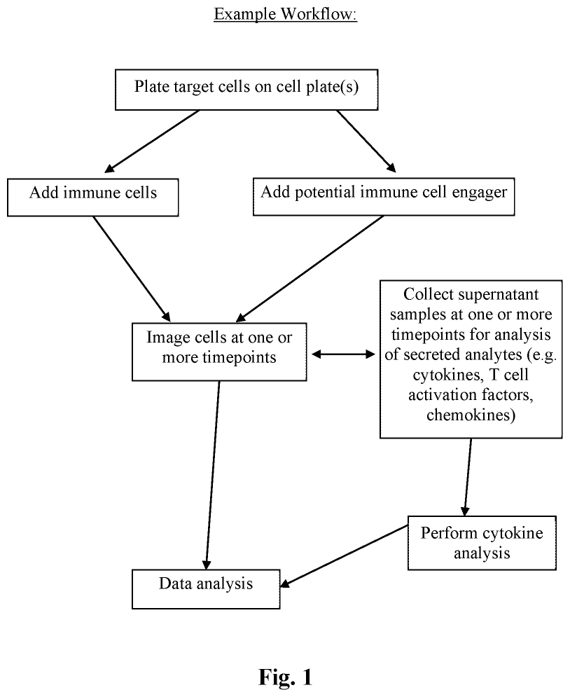

In some embodiments, assays herein may follow a workflow as depicted in FIG. 1, wherein target cells such as tumor cells are added to wells of a cell plate followed by immune cells and potential immune cell engager. In other cases, the order of addition may differ, e.g., immune cells may be added before tumor cells, or a potential immune cell engager may already be included in wells of a plate before cells are added, or ingredients may be added relatively simultaneously, etc.

In some embodiments, the workflow comprises plating target cells, in some cases with an automated cell culture apparatus, onto plates, such as 96 well or 384 well plates. In some embodiments, cells may be plated at a relatively uniform volume and/or concentration per well. For example, in some embodiments, cells are plated at, for example 1000-50,000 cells/well, or 5000-30,000 cells/well, or 5000-20,000 cells/well, 1000-20,000 cells/well, 1000-10,000 cells/well, 1000-5000 cells/well, 5000-10,000 cells/well, or 8000-15,000 cells/well, or 8000-12,000 cells/well, or 1000 cells/well, or 2000 cells/well, or 5000 cells/well, 7000 cells/well, 8000 cells/well, 9000 cells/well, 10,000 cells/well, 11,000 cells/well, 12,000 cells/well, or 15,000 cells/well. In some embodiments, cells are plated at, for example, 5-50 μL/well, 10-50 μL/well, 20-50 μL/well, 20-40 μL/well, 20-30 μL/well, 20-25 μL/well, 25-30 μL/well, 30-50 μL/well, 30-40 μL/well, 25-35 μL/well, 10 μL/well, 15 μL/well, 20 μL/well, 25 μL/well, 30 μL/well, 35 μL/well, or 40 μL/well. In some embodiments, cells may be added to a cell plate using an automated cell counter and/or liquid handling system, for example to ensure relatively uniform distribution of cells in each well of a plate. In some embodiments, plated cells may be incubated an automated cell culture apparatus, such as SelecT™ (Sartorius).

After plating of target cells, immune cells and/or potential immune cell engager molecules may be added to the wells of the cell plates, for instance, at particular concentrations. For example, in some embodiments, potential immune cell engager molecules and/or immune cells may be added to the plates at specific cell number and concentrations, and for example at increasing or decreasing concentrations. For example, in some embodiments, immune cells are added at, for example 1000 to 50,000 cells/well, or 5000-50,000 cells/well, or 10,000-40,000 cells/well, 10,000-30,000 cells/well, or 5000-20,000 cells/well, or 1000-20,000 cells/well, 1000-10,000 cells/well, 1000-5000 cells/well, 5000-10,000 cells/well, or 8000-15,000 cells/well, or 8000-12,000 cells/well, or 1000 cells/well, or 2000 cells/well, or 5000 cells/well, 7000 cells/well, 8000 cells/well, 9000 cells/well, 10,000 cells/well, 11,000 cells/well, 12,000 cells/well, or 15,000 cells/well. In some embodiments, immune cells are added at, for example, 5-50 μL/well, 10-50 μL/well, 20-50 μL/well, 20-40 μL/well, 20-30 μL/well, 20-25 μL/well, 25-30 μL/well, 30-50 μL/well, 30-40 μL/well, 25-35 μL/well, 10 μL/well, 15 μL/well, 20 μL/well, 25 μL/well, 30 μL/well, 35 μL/well, or 40 μL/well. In some cases, they may be added at several different amounts in different wells, for example, to compare the effects of different immune cell to tumor cell ratios or to titrate immune cells against tumor cells. Similarly, potential immune cell engager molecules may be added at specific concentrations in particular wells, for example, to determine effective concentrations of the molecules that lead to tumor cell engagement by immune cells and that lead to tumor cell killing. For example, potential immune cell engager molecules may be added at 1 nM to 10 μM concentrations in some embodiments, for example, using 2.5 nanoliter-10 μL volumes, such as 5 nL-1 μL 1 nL-100 nL, 10 nL-1 or 100 nL-10 μL. In some embodiments, the potential immune cell engager may be added at 1 nM to 1 such as 1 nM-100 nM, 10 nM-1 μM, 100 nM-10 μL 1 nM-10 nM, 10 nM-100 nM, 100 nM-1 μM or 1 μM-10

In alternative embodiments, immune cells may be plated in the first step and then target cells may be added to the immune cells on the plate.

Addition of further cells and/or potential immune cell engager molecules may be conducted at low volumes using acoustic volume dispensing or equivalent methods, for example. An Echo acoustic dispenser (Beckman Coulter) is an exemplary apparatus allowing for acoustic-controlled volume dispensing. In some embodiments, potential immune cell engagers may be added to a cell plate before or after cells are added. In some cases, depending on the requirements of the apparatuses used, potential immune cell engagers may be added before all of the cells are added. In some cases, this may be due to equipment limitations. For example, acoustic controlled volume dispensers may require plates to be manipulated in a way that limits the volume of material that may be present in each well. Thus, where this is the case, immune cell engagers may be added before each well has been filled with all of the cells for co-culturing.

Following addition of a potential immune cell engager to an immune cell and tumor cell population, the plates may be incubated at various temperatures for varying degrees of time in order to assay one or multiple parameters associated with engagement of immune and tumor cells, immune cell activation, and/or tumor cell killing. Exemplary parameters that may be assayed include target cell death, target cell apoptosis, and changes in cytokine concentrations associated with immune cell activation and/or target cell killing. In some cases, the methods herein may also be combined with flow cytometry analysis to determine changes in immune cell populations in the sample wells. In some embodiments, each well may support more than one type of assay, such as a cell death and/or apoptosis assay, as well assays of changes in one or more cytokine concentrations.

In some embodiments, a single measurement of a parameter, such as related to cell death or apoptosis, may be obtained, so as to obtain an endpoint measurement for that parameter. In some embodiments, the assays may be performed at more than one point in time in order to determine the kinetics of cell death, apoptosis, and cytokine concentrations, for example. In some cases, results may be quantitated. Thus, in some embodiments, parameters such as kinetics of cell killing (e.g., KT50) may be determined. In some cases, an assay may be determined at multiple concentrations of potential immune cell engager and/or immune cells, for example, to obtain an EC50 for the engager or immune cells.

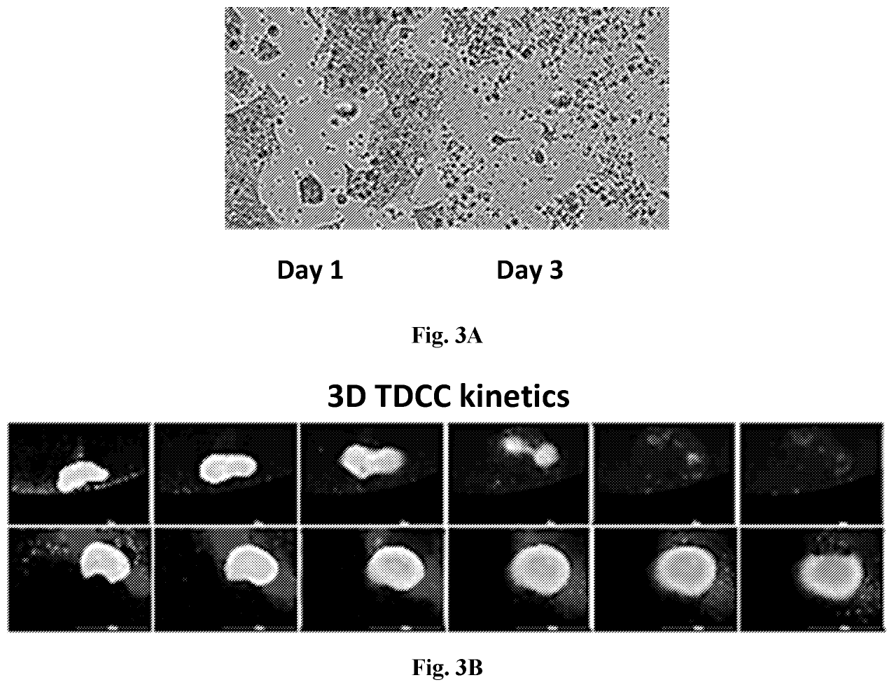

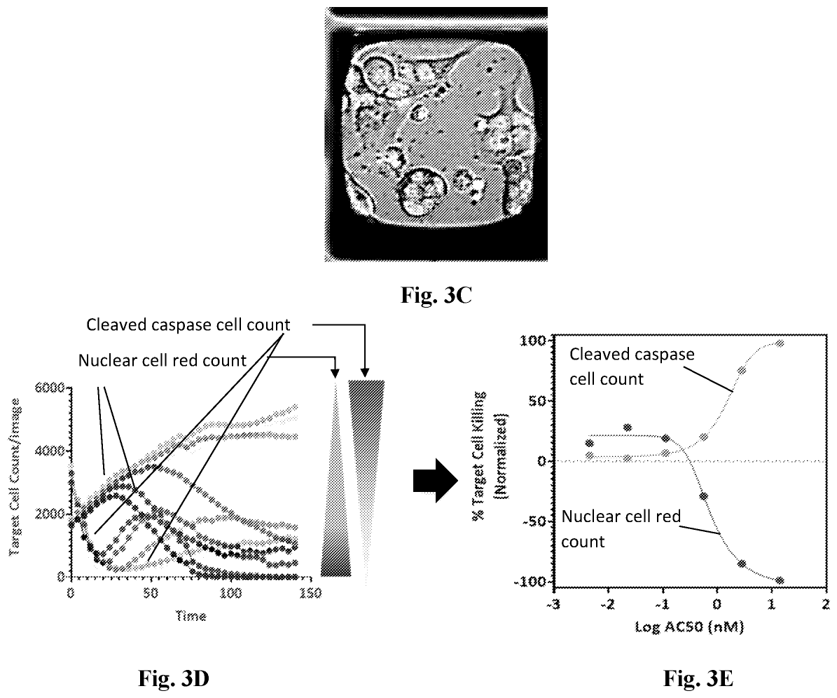

Parameters that may be assayed include death of target cells, for example, by transducing or labeling target cells with a nuclear fluorescent protein or dye and recording changes in the intensity of the dye label upon exposure to immune cells with or without addition of a potential immune cell engager (e.g. changes in fluorescence for a fluorescent dye). In some embodiments, the dye is introduced into a cell by transduction, for instance with lentivirus or another transduction method. In some embodiments, a nuclear fluorescent protein, such as NucLight™ Red or Green may be used (e.g. Incucyte® NucLight™ lentivirus introduced or rapid red or green fluorescencre protein, Sartorius). (See, e.g., FIGS. 3A-E, 7A-7F, and 8A-8C for examples.) Transduced nuclear dyes, for example, may include fluorescent nuclear proteins. In some embodiments, labeling a target cell with a transduced dye system may be preferable to a general cell stain, as a nuclear fluorescent protein produced from such a system may be less likely to bleed from target cells to immune cells in comparison to a general cell membrane or cytoplasmic stain. In some embodiments, cell death may be monitored by reading the signal from the label over time, thus allowing determination of the rate of cell death as well as the extent of the cell death, e.g., as the percentage of cells killed. Parameters may also include time to 10%, 25%, 50%, 75%, or 90% or 100% cell death, for example. In some embodiments, Incucyte® software or similar programs may be used to identify target cell number with specific fluorescence intensities. In some cases, segmentation parameters can be optimized to best identify cell number changes over time.

In addition to or as an alternative to tracking cell death in the assay, apoptosis of target cells may also be tracked, for instance, by using a different color dye. For example, in some embodiments a caspase 3/7 dye system (e.g. a caspase 3/7 green or red dye) may be used to track apoptosis activity. (See, e.g., Incucyte® caspase 3/7 green or red fluorescent dye reagents from Sartorius; and see FIGS. 3C-3E.) For example, caspase 3/7 dyes may be used to detect cells undergoing apoptosis mediated by caspase 3/7, as the dye molecules, which may penetrate the cell membrane, are activated and emit a fluorescent signal only after they are cleaved by caspase 3/7. The dye then is able to intercalate into DNA in the cell. Accordingly, caspase 3/7-mediated apoptosis causes an increase in fluorescence that may be measured over time in the assays herein. Thus, apoptosis, like cell death, may be determined kinetically in some embodiments, for instance to determine a KT50 or other values associated with the apoptosis process. In some embodiments, apoptosis may be monitored by reading the signal from the label over time, thus allowing determination of its rate and extent. Parameters may also include time to 10%, 25%, 50%, 75%, or 90% 100% of the maximum apoptosis signal, for example. In some embodiments, both apoptosis and cell death may be measured in the same wells, using two different fluorescent signals and dyes, and their rates and extents compared.

In some embodiments, measurements from such labels may be made by incubating cell plates in an image analyzer designed for such purpose, such as an Incucyte® live cell imager (Sartorius).

In some embodiments, samples of the supernatant from the wells are removed at particular points in time for analysis of changes in concentration of molecules secreted from cells, such as immune cell activation markers, cytokines or chemokines. For example, small volumes such as 1-10 microliters, 2-10 microliters, 2-8 microliters, 4-8 microliters, 2, 4, 5, 6, 7, 8, or 10 microliters may be removed from the supernatant at least once during, prior to, or after incubation of target cells and immune cells with or without a potential immune cell engager. Supernatant samples may also be collected at regular time intervals for kinetic assays of changes in the concentrations of secreted factors. In some cases, a total supernatant volume comprising no more than 50% of the original volume of a sample or well may be removed for these analyses. For example, removing too much supernatant might also remove growth media components that the cells need to retain normal growth. Thus, in other words, if the volume of the co-cultured cells and additional ingredients such as potential immune cell engager, and any associated media, etc., upon original addition of ingredients adds up to a particular volume, in some cases, no more than 50% of that original volume total may be removed for these analyses. In some cases, no more than 40%, or no more than 30% or no more than 20% is removed for these analyses. In some embodiments, supernatant is removed at least twice or at least three times during incubation of the cells. And in some such cases, no more than 50%, no more than 40%, no more than 30% or no more than 20% of the original volume is removed during all of the combined supernatant collections.

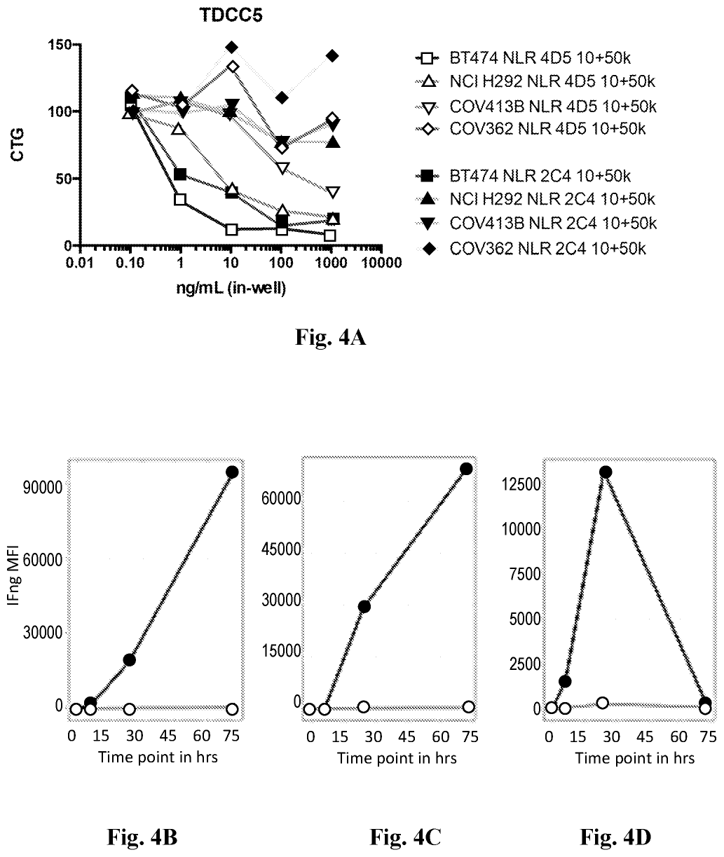

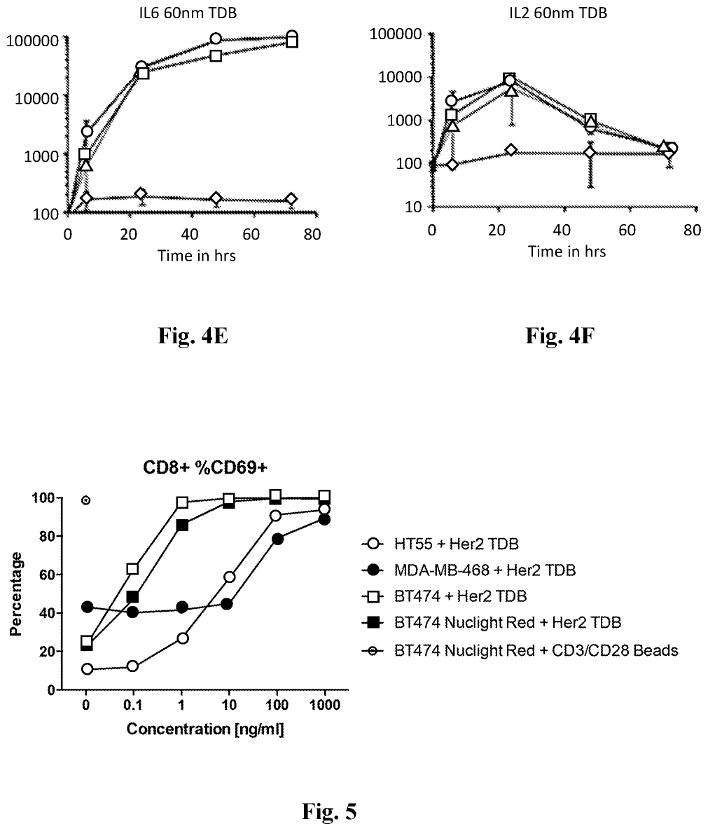

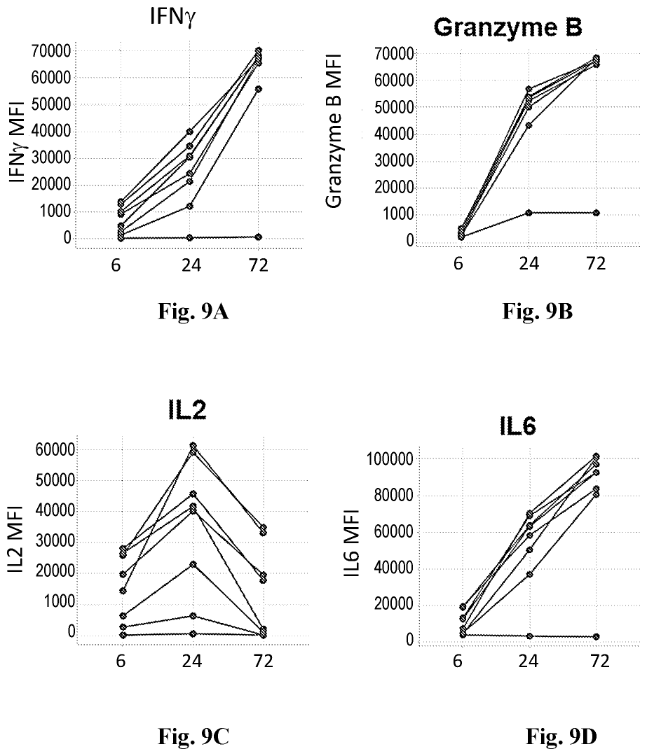

In some embodiments, concentration of one or more analytes found in the supernatant samples, such as cytokines or other T cell activation related factors (Granzyme B) or chemokines may be assayed, for example, using multiplexed beads or arrays. For example, multiplexed assays for secreted proteins such as cytokines, T cell activation factors, and chemokines may be employed, which in some cases use beads with different color labels that detect binding of each particular secreted protein to beads specifically recognizing that protein. For example, an array or bead or rod may contain molecules binding to several different cytokines, each bead or array or rod with a unique color label combined with the binding label allow quantitation of the analyte, allowing binding of the cytokines to the labeled binding agent to be tracked in a multiplexed fashion. In some embodiments, cytokine analysis may be conducted using a Luminex FlexMap 3D® imaging system. In some embodiments, cytokines and other analytes that may be assayed include, for example, perforin, granzyme b, interferon gamma (IFNγ), IL-10, IL-2, IL-6, IL-8, MIPla, MIP1b, TNF-alpha (TNFα). (See, e.g., FIGS. 4B-4F and 9A-9D.) Other analytes that may be assayed in some embodiments include, for example, human growth hormone (HGH), N-methionyl human growth hormone, and bovine growth hormone; parathyroid hormone; thyroxine; insulin; proinsulin; relaxin; prorelaxin; glycoprotein hormones such as follicle stimulating hormone (FSH), thyroid stimulating hormone (TSH), and luteinizing hormone (LH); epidermal growth factor (EGF); hepatic growth factor; fibroblast growth factor (FGF); prolactin; placental lactogen; mullerian-inhibiting substance; mouse gonadotropin-associated peptide; inhibin; activin; vascular endothelial growth factor; integrin; thrombopoietin (TPO); nerve growth factors such as NGF-alpha; platelet-growth factor; transforming growth factors (TGFs) such as TGF-alpha and TGF-beta; insulin-like growth factor-I and-II; erythropoietin (EPO); osteoinductive factors; interferon beta (IFNb), colony stimulating factors (CSFs) such as macrophage-CSF (MCSF); granulocyte-macrophage-CSF (GM-CSF); and granulocyte-CSF (G-CSF); interleukins (ILs) including IL-10, IL-2, IL-6, IL-8, as well as IL-1, IL-lalpha, IL-1beta, IL-3, IL-4, IL-5, IL-7, IL-9, IL-11, IL-12 (p′70), IL-12 (p40), IL-13, IL-15, IL-17/17A, IL-17C, IL-17D, IL-17F, IL-18, IL-20, IL-21, IL-22, IL-23 (p19), IL-27, IL-35, TNF-beta, GFAP, MMP1, MMP2, MMP3, MMP7, MMP9, MMP10, MMP12, TNF-R1, TNF-R2, VEGF-A, lymphotoxin beta, CCL1, CCL2 (MCP-1), CCL3 (MIP-1 alpha), CCL4 (MIP-1 beta), CCL5 (RANTES), CCL6, CCL7 (MCP-3), and CCL8 (MCP-2); and other polypeptides.

In some embodiments, where multiple parameters are assayed, for example, kinetically, the kinetics may also be compared, for example, to obtain a more complete picture of the impact of a potential immune cell engager on the system.

In some embodiments, where a range of concentrations of a particular potential immune cell engager is assayed, an EC50 or IC50 measurement may be obtained in order to assay the dose-response relationship of the potential immune cell engager and the effect being measured. For example, the slope or the area under the curve (AUC) for a particular change in cytokine concentration, or for cell death may be obtained at each of several concentrations of the potential immune cell engager, from which an EC50 or IC50 may be calculated for the potential immune cell engager. In some embodiments, this may allow the potency of different potential immune cell engagers to be compared, or the potency of a single engager to be compared for different tumor cell samples.

Kinetic traces can also be converted into dose response curves to determine an EC50 for each molecule potency or cytokine profile. The rate it takes to kill 50% of the cells, for example, can be compared to rank speed of killing. The maximum activity of the killing can be determined to show the maximum percentage of cells that can be killed. The difference in the minimum and maximum activity can be used to confirm the maximum activity. The endpoint levels of cell killing-related parameters, such as ATP activity for example, can be compared to the % apoptosis or killing, for example, at the last time point assessed or at intermediate time points. In some cases, immune cells can be removed from the 384-well or 96-well plates and characterized using flow cytometry.

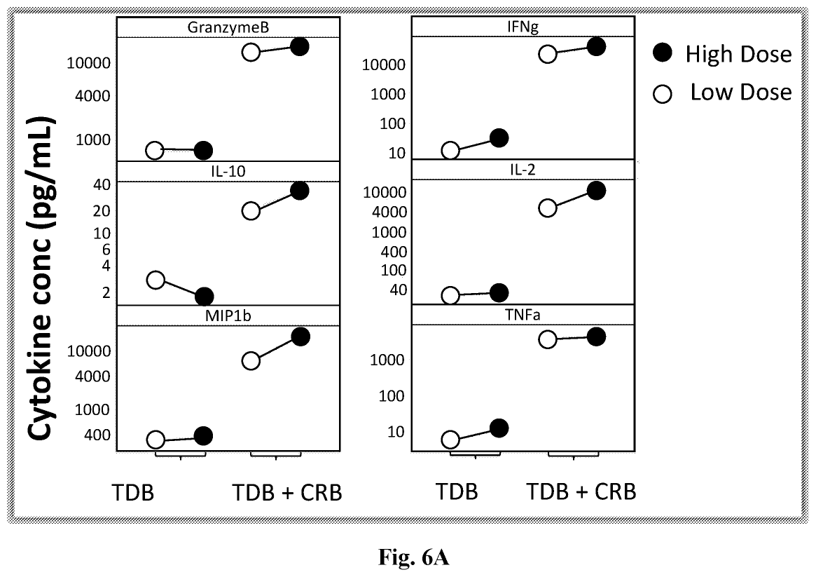

In some embodiments, cells in one or more wells may be further characterized by flow cytometry. In some embodiments, immune cells may be evaluated by flow cytometry to characterize cell types and/or assess particular cellular activation markers. T cells may be assessed for changes in the level of T cell markers such as CD3, CD4, CD8, CD69, HLA-DR, and/or CD25. (See, e.g., FIG. 5.) Additional markers that may be assessed by flow cytometry include, for example, CD11b, CD19, CD56/NCAM-1, CD94, CD122/IL2 receptor beta, CD127/IL7 receptor alpha, CD152, Fcγ RIII, CD16, KIR family receptors, NKG2A, NKG2D, NKp30, NKp44, NKp46, NKp80, IFNγ, TNF, EOMES, CXCR3, IL2, IL4, IL10, IL12, IL18, STAT1, STAT4, STATS, FOXP3, CCR4, Thus, flow cytometry may allow determination of how potential immune cell engagers impact T cell activation, for example. Immune cells, such as PBMCs, for example, may be assessed for percentage of CD3+, CD4+, and CD8+ cells or for other markers as listed above.

In some embodiments, further assays related to target cell killing may also be performed in the systems herein. In some embodiments, a Cell Titer Glo® (CTG) ATP assay (Promega) may also be conducted. Such assays determine the degree of viability of cells on a plate well by determining the amount of ATP present in the well, since ATP is an indication of active cellular metabolism. (See FIG. 4A.) Other exemplary assays that are compatible with the methods and systems herein include luciferase reporter assays to track particular gene expression, enzyme-linked immunospot (ELISpot™) assays (e.g. from Mabtech, Inc., Cincinnati, OH) to assess the amount of cytokine releasing cells in particular wells, and lactate dehydrogenase (LDH) release assays for example as a further assay of cellular cytotoxicity.

In some embodiments, the above workflows, for example, as shown in FIG. 1, may be performed in a period of 1-15 days, such as 1-10 days, 1-5 days, or 1-3 days. For example, in some embodiments, the cells are incubated together with the potential immune cell engager for a period of 1-15 days, such as 1-10 days, 1-5 days, or 1-3 days, or in 1, 2, or 3 days, depending on the growth rates of the co-cultured cells and/or the potency of the potential immune cell engager. Thus, for example, some embodiments allow up to hundreds of different combinations of tumor cell, immune cell, and potential immune cell engager, optionally under a variety of conditions or in the presence of other molecules, to be assayed in a short space of time such as 1-15, 1-10, 1-5, or 1-3 days, and at different ratios of cell and immune cell engager reagents, or using cell samples from different donors at a variety of concentrations.

Potential immune cell engagers that may be evaluated in assays herein include, for example T cell dependent bispecific antibodies and costimulatory receptor bispecific antibodies (TDBs and CRBs). In some embodiments, potential immune cell engagers such as TDBs may bind to a molecular target expressed on T cells, such as CD3. Alternatively or additionally, potential immune cell engagers may bind to another immune cell surface marker such as CD56/NCAM-1, CD94, CD122/IL2 receptor beta, CD127/IL7 receptor alpha, Fcγ RIII, KIR family receptors, NKG2A, NKG2D, NKp30, NKp44, NKp46, or NKp80, for example, and also to a molecular target expressed on tumor cells. Example tumor cell targets include, for instance, HER2, CD20, PSCA, CD19, Flt3, CD33, EGFR, MCSP, CEA, EpCAM, Steapl, FcRH5, DLL3, Ly6G6D, LyE, Napi3b, muc, CD22, immature laminin receptor, TAG-72, HPV E6, E7, BING-4, calcium-activated chloride channel 2, CCNB1, 9D7, EphA3, mesothelin, SAP-1, Survivin, a member of the BAGE, CAGE, SAGE, or XAGE family, NY-ESO-1/LAGE-1, PRAME, SSX-2, melan-A/MART-1, Gp100/pmel 17, tyrosinase, TRP-1/-2, P.polypeptide, MC1R, beta-catenin, BRCA1, BRCA2, CDK4, CML66, fibronectin, MART-2, and the like, depending upon the cell type to be targeted. Instead of T cells, in some embodiments bi-specific molecules such as antibodies may be designed to link other immune cells to tumor cells, e.g., NK cells, and then to a target molecule on a target cell.

CRBs may bind to an immune cell target such as CD28, CD27, OX40, 4-1BB (CD137), CD30, Tim1,2,3, GITR, CTLA4, BTLA, LFA-1, PD1, NKG2D, B&-1,2, LIGHT, or ICOS, as well as to a tumor target.

Any type of cell that may be targeted for destruction by an immune cell may be a target cell in the assays herein. In some cases, the target cell is a primary cell. In some cases, it is a tumor cell. In some embodiments, tumor cells may be cultured cells, such as cultured human tumor cells. In some embodiments, target cells may be pre-treated with a nuclear cell transduction reagent so that they express a fluorescent protein, e.g., in the nucleus, such as via lentivirus transformation. In some embodiments, target cells may be derived directly from a patient, such as tumor cells or suspected tumor cells from a biopsy. In some embodiments, tumor cells may be solid tumor cells. In other embodiments, tumor cells may be non-solid tumor cells, such as lymphoma or leukemia cells. In some embodiments, tumor cells may be from breast cancer, lung cancer (including small cell lung cancer or non-small cell lung cancer, adenocarcinoma of the lung, and squamous carcinoma of the lung), prostate cancer, testicular cancer, penile cancer, esophageal cancer, tumors of the biliary tract, brain cancer (including glioblastoma), colorectal cancer, colon cancer, rectal cancer, kidney cancer (including renal cell carcinoma), liver cancer (hepatoma), adrenal cancer, cervical cancer, uterine cancer, endometrial cancer, vulval cancer, salivary gland carcinoma, squamous cell cancer of the head and neck, leukemia, lymphoma, lymphoid cancer, ovarian cancer, pancreatic cancer, bladder cancer, skin cancer such as melanoma, or urinary tract cancer.

In some embodiments, immune cells are T cells, such as CD4+ T cells or CD8+ T cells or Pan T cells. In other embodiments, immune cells are PBMCs. In other embodiments immune cells are NK cells. In some cases, immune cells used in the assays may include a mixture of cells, such as a mixture of T cells, B cells, and/or NK cells. In some embodiments, immune cells are derived from a particular donor. For example, the assays herein may be used to compare the tumor cell engagement of immune cells from different donors in the presence of different immune cell engagers. Thus, in some embodiments, assays herein could be used to screen potential immune cell engagers against the immune cells and/or tumor cells obtained from an individual donor. In other cases, assays herein may be used to test potential immune cell engagers on co-cultured cells using immune and/or target cells taken from a two or more donors, for example, to compare the activity of engagers over several different co-cultured cell populations.

In some embodiments, assays herein may include testing a potential immune cell engager against more than one type of target cell. In some embodiments, a potential immune cell engager may be tested against immune cells from more than one donor, or of more than one type (e.g. CD8+ T cells vs pan T cells, etc.). In some embodiments, potential immune cell engagers may be tested at different ratios of immune cell and target cell. In some such cases, one cell type may be titrated against the other, for example. In some embodiments, the potential immune cell engager may be titrated against a constant amount and ratio of target and immune cells, and further, against a set of specific combinations and ratios of target cells and immune cells. For example, the use of multi-well plates and automated processing coupled with small volumes of reagents can allow multiple different tests such as the above to be run in parallel in one or more cell plates. In some embodiments, such data may also be collected within a short space of time such as from 1-15 days, 1-10 days, 1-5 days, 1-3 days, or in 1-2 days, depending on the speed of cell growth and target cell killing in the assays.

### Exemplary Systems

The present disclosure also encompasses systems for conducting the methods described herein. In some embodiments, for example, a system may be capable of performing two or more assays herein in tandem in an automated fashion. In some embodiments, a system may be capable of dispensing reagents for co-culturing of immune and target cells, and adding potential immune cell engager and/or other reagents to the co-culture. In some embodiments, the system may be capable of dispensing reagents, incubating and monitoring co-cultured cells, and analyzing parameters herein such as changes in fluorescence from one of more cell stains, and/or changes in supernatant concentrations of protein markers such as cytokines. In some embodiments, systems herein may be partially or fully automated.

In some embodiments, systems herein may comprise, for example, cell culture plates, such as 96-384 well multi-well plates, at least one liquid handling dispenser for adding cells and/or reagents to cell plates, at least one imager apparatus for incubating and monitoring fluorescence levels in wells of a cell plate, and/or an apparatus for removing supernatant from wells of cell plates for analysis of analytes such as immune cell markers, cytokines, and chemokines in the supernatant.

In some embodiments, systems herein also comprise data analysis software for performing end point and/or kinetic analyses of parameters herein.

### EXAMPLE

FIG. 1 shows an example workflow for an exemplary high throughput, multiparametric, automated system for conducting assays herein. Fluorescent red or green Nuclight™ tumor cells are maintained, optionally using an automated cell culture apparatus (e.g. SelecT™ from Sartorius), and plated either manually or automatically, for example using an automated liquid dispenser (e.g. Certus, Tecan or Agilent (Bravo)) onto a 96 or 384 well plate. A potential immune cell engager is added along with immune cells to the plate wells, optionally using an acoustic dispenser (Echo, Beckman Coulter). Green or red nuclear fluorescence proteins (e.g., NucLight™, green fluorescent protein (GFP), mCherry, TurboGFP) or dyes (e.g. caspase 3/7 dyes, Sartorius) are tracked over time in an imaging apparatus (e.g., Incucyte®, Sartorius). Optionally, cytoplasmic or membrane dyes (e.g., Cytolight™) may be used to stain and differentiate immune cells from tumor cells to improve quantitation of killing for only the tumor cells and to exclude death signal from dying immune cells, to obtain kinetics of target cell death and apoptosis. Supernatants from the wells are optionally collected for analysis of secreted analytes such as cytokines, for example, using magnetic beads (available from Luminex) using a FlexMap 3D® reader (Luminex) to determine concentrations of various analytes simultaneously from the supernatant samples. Data are analyzed using, for example Spotfire® (TIBCO) and/or Genedata (Basel, CH) software packages.

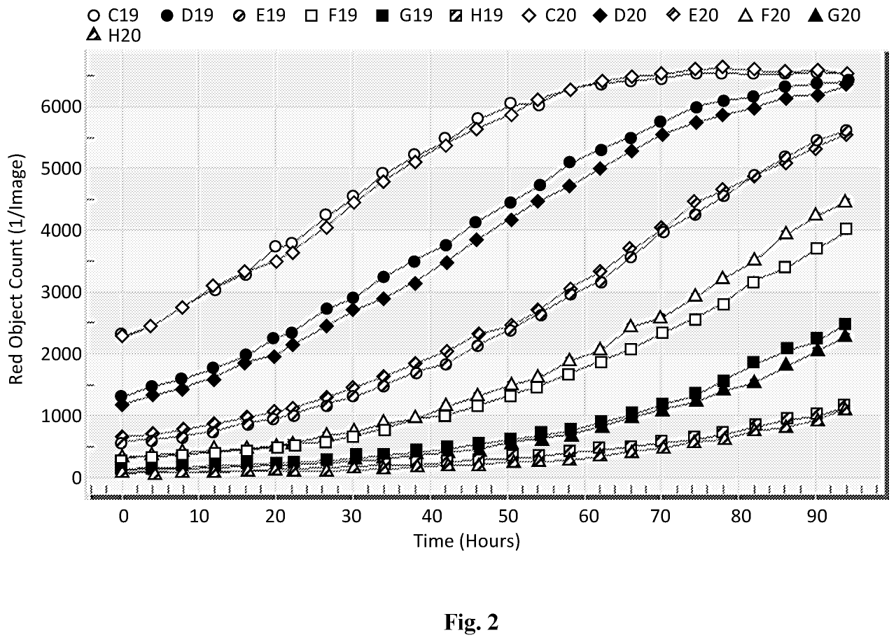

To run methods according to FIG. 1, cell density used for plating cells was optimized, as shown in FIG. 2. To ensure uniform cell plating of fluorescent cells, first a suitable fluorescent nuclear protein marker, introduced via lentivirus transduction, and that is unstable when cells die and loses its signal was chosen, followed by optimization of the protocol (i.e., ensuring sufficient lentivirus transduction for integration of the fluorescent protein). The cells were then counted and plated into multiple wells, in duplicate (C19 and C20; D19 and D20, E19 and E20, shown in FIG. 2, are each duplicates, etc.). Samples C, D, E, F, G, and H, shown in FIG. 2, differ in the number of cells, with C having 2-fold more cells than D, and D having 2-fold more cells than E, etc. Imaging was performed every 4 hours and cells were quantitated using the red object count. An assay as described herein may be used to select an optimal cell density for the assays, and may for example be a density in which cells proliferate over time but the growth curve of the cells does not reach its maximum during the length of the planned assay duration.

Tumor cells and immune cells were co-cultured in the presence of a T cell dependent bispecific antibody (TDB). FIG. 3 shows changes in fluorescence of tumor cells over time following addition of immune cells and a T cell dependent bispecific antibody (TDB) to tumor cells, reflecting killing and apoptosis of tumor cells. FIG. 3A shows the level of nuclear fluorescence intensity (NucLight™ red) after 1 or 3 days after addition of immune cells and TDB. As shown in FIG. 3B, co-cultured tumor cells were stained green and appear as a 3D spheroid, while Cytolight™ stained immune cells were stained red. The tumor and immune cells were co-cultured and incubated over a period of time (left to right panels) with a TBD (top row) or without a TBD (bottom row). As can be seen, presence of TBD (top row) causes loss of green staining over time due to death of tumor cells, in comparison to lack of TBD (bottom row), in which staining does not change significantly over time. Migration and penetration of the red immune cells into the tumor spheroid were captured and were quantitated in addition to the tumor cell loss. FIG. 3C shows single cell killing activity in a 86,400 sub-well of a micro-well 384-well plate, showing individual tumor cells (red staining) and caspase 3/7 green fluorescent label (green spots). FIG. 3D shows changes in intensity of nuclear red fluorescent dye and caspase 3/7-dependent green fluorescent dye in tumor cells in the presence of immune cells and an immune cell engager. Tumor cell death causes loss of the red nuclear fluorescence, while increased apoptosis causes an increase in intensity of the caspase 3/7-dependent green fluorescent stain. The co-culture was treated with a dose titration of TDB with one tumor cell line and one donor immune cell line at a 1:1 ratio. FIG. 3E shows the dose response curve (DRC) generated using the kinetic traces of the different treatment concentrations.

Various other parameters associated with engagement and killing of tumor cells were also assessed in the assays, such as endpoint killing and changes in cytokine concentrations. FIG. 4A shows tumor cell killing based on metabolism (ATP) readout across 4 different tumor cell lines (BT474, NCIH292, COV413B, and COV362) in the presence of 2 different TDBs (NLR 4D5 and NLR 2C4). FIGS. 4B, 4C, and 4D show changes in concentrations of IL-6, IFNγ, and IL-2, respectively, in supernatants taken from wells (upper curves), in comparison to a non-tumor target control TDB (bottom curves). FIGS. 4E and 4F show MFI signal of 2 analytes (IL-6 and IL-2) treated with 60 nM TDB over time across 4 immune cell donors. Assays were performed in an 8-plex system from Luminex and data plotted over time.

FIG. 5 shows a determination of the percentage of CD8+CD69+ T cells by flow cytometry of immune cells isolated from 384-wells, performed at the end of the image collection for 4 cell lines titrated with TDB or bead stimulation. Immune cells were stained with fluorescent antibodies recognizing CD8 and CD69 markers on the surface of the immune cells. Upregulation of T cell activation markers coincided with the higher expressing tumor target expressing cell lines.

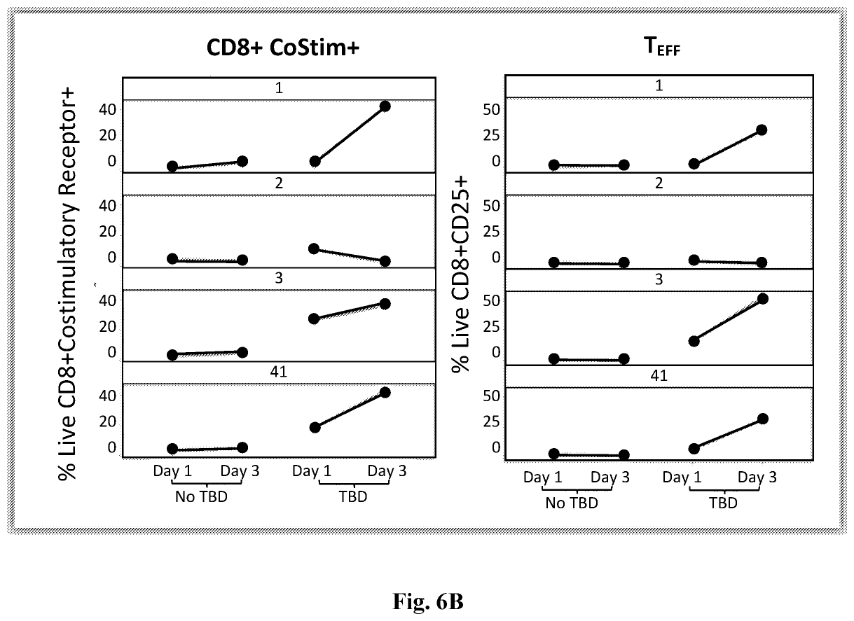

Concentrations of granzyme B, IL-10, MIP1b, IFNγ, IL-2, TNFα at high (dark circles) and low (light circles) doses of TDB in a co-cultured cell supernatant were also assessed, with TDB alone or a combination of TDB and a CRB (costimulatory receptor bispecific antibody). (See FIG. 6A.) FIG. 6B shows, in the left panel, differences in the percentage of CD8+ and costimulatory receptor+(CoStim+) T cells after incubation with no TDB and with a TDB after 1 and 3 days, and in the right panel, the percent CD8+CD25+ T cells (Teff) with or without TDB after 1 and 3 days.

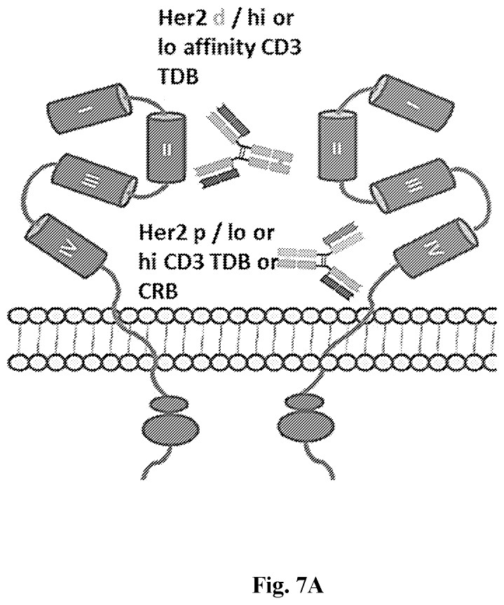

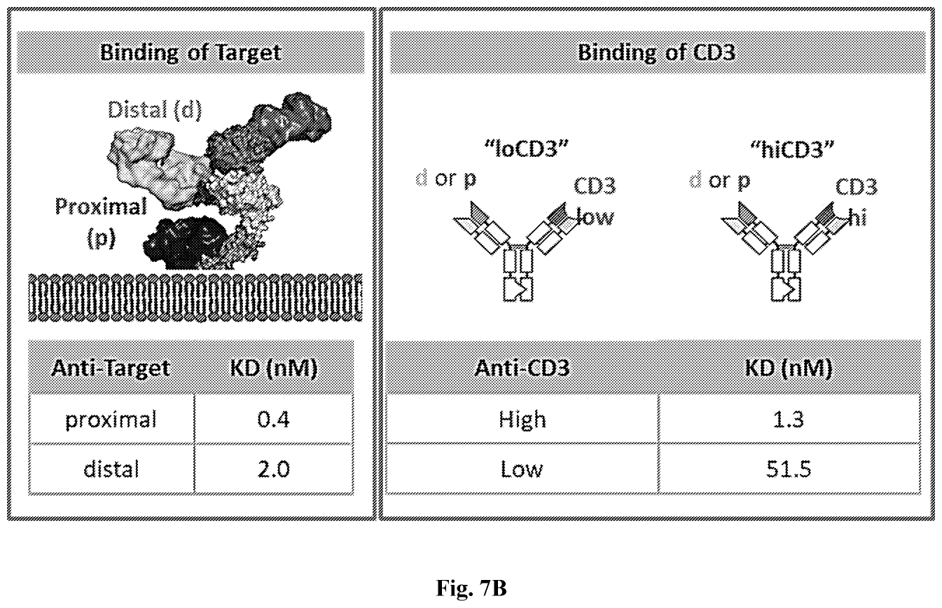

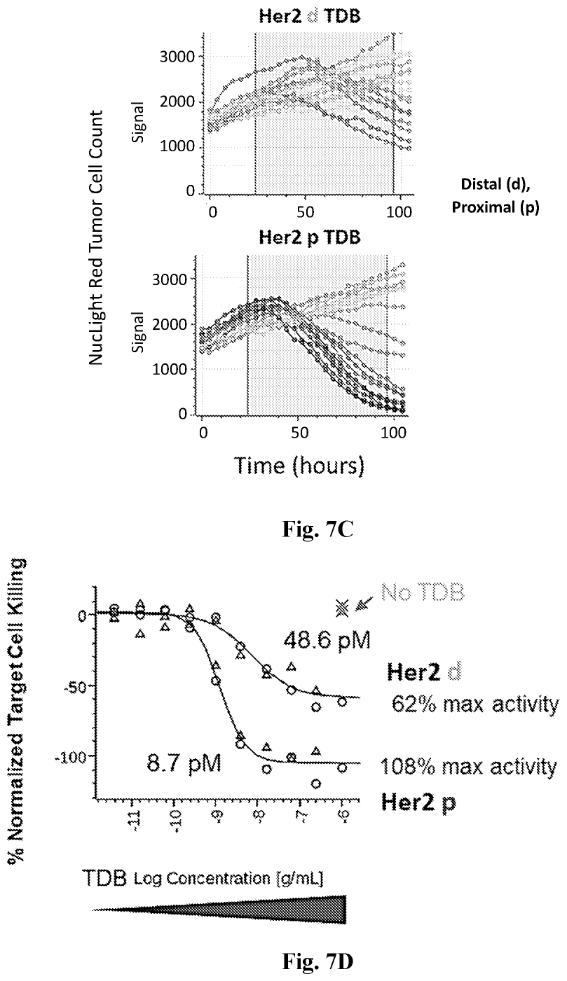

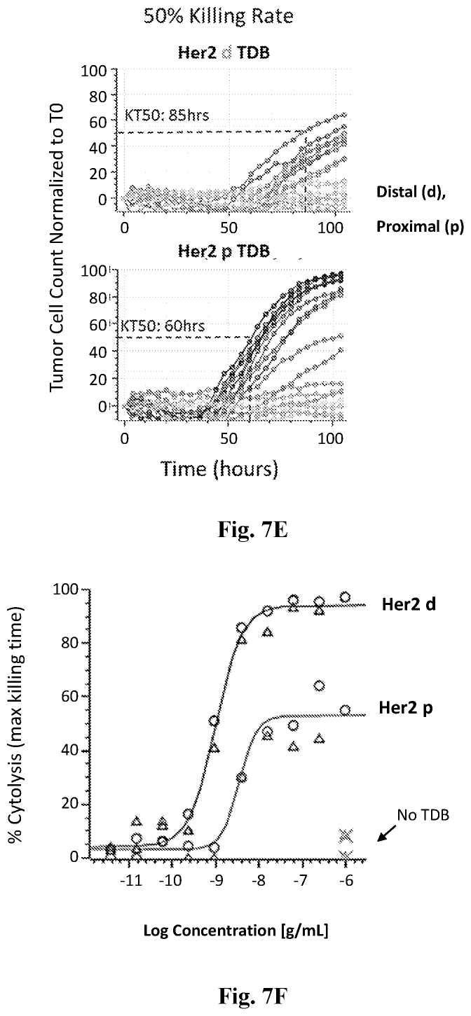

FIG. 7A shows a schematic of different TDBs binding to Her2 in either a proximal (p) or distal (d) fashion, and binding to CD3 with either high (hi) or low (10) affinity. FIG. 7B provides the relative affinities of the individual anti-Her2 or anti-CD3 arms of the TDBs. FIG. 7C provides kinetic traces for the 2 TDBs, showing greater loss of cells for the higher affinity TDB treatment. FIG. 7D shows the conversion of the kinetic traces into a dose response curve for the 2 TDBs. FIG. 7E shows calculation of time it takes to kill 50% of the tumor cells for the 2 TDBs, indicating they have different rates of killing. FIG. 7F shows the dose response curves generated from the % cytolysis traces in 7E.

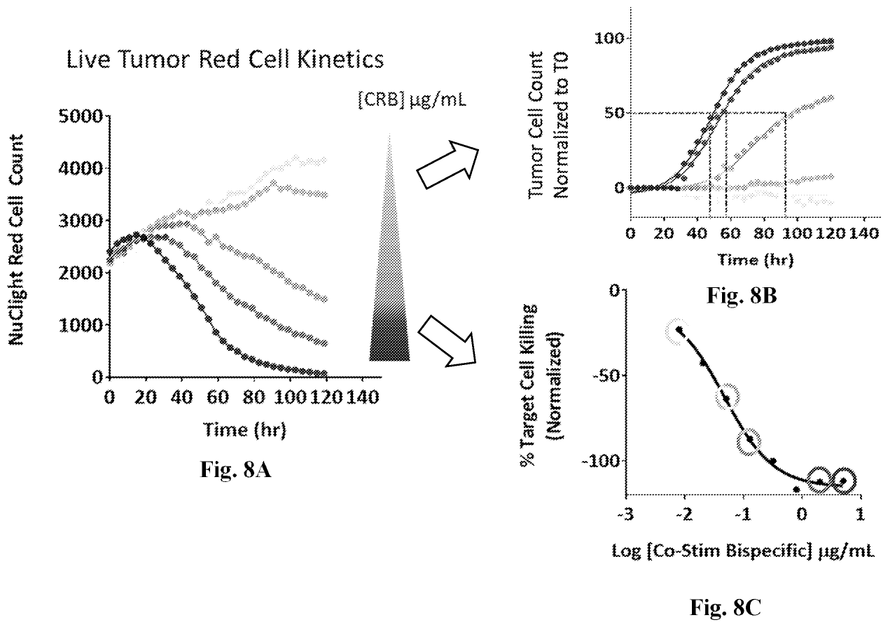

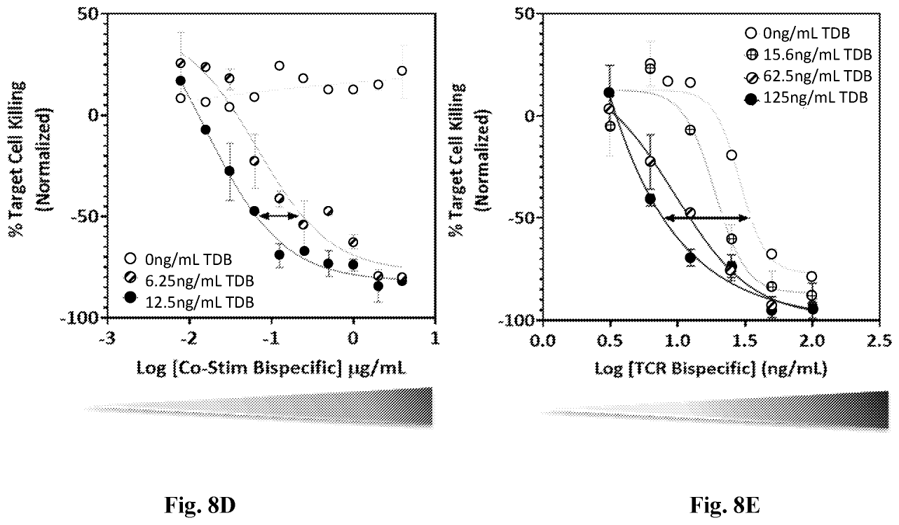

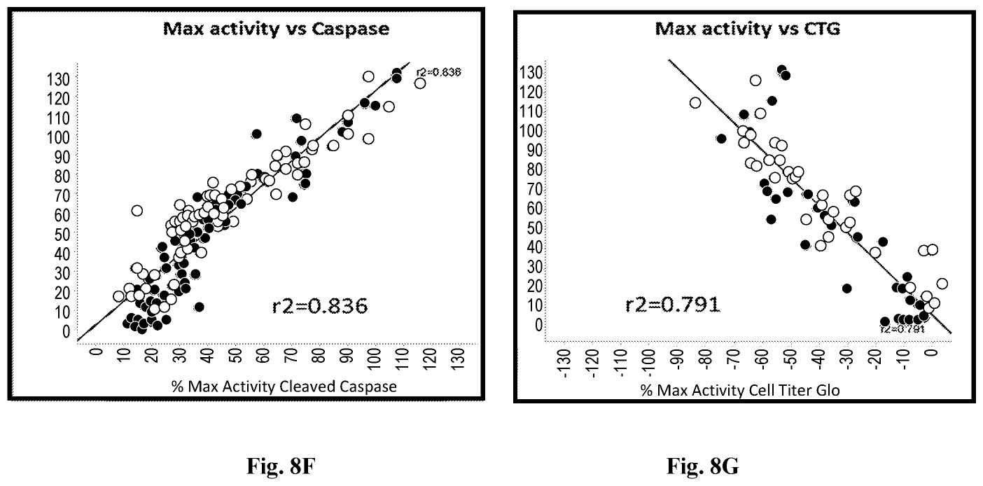

FIG. 8A shows titration of a CRB co-dosed with a fixed amount of TDB. Darker curves represent higher relative concentrations of CRB to TDB. FIG. 8B shows calculation of the KT50 rates for the different treatment concentrations. FIG. 8C shows DRC calculation from the individual traces. FIG. 8D shows the percentage of target cell killing (normalized) for a titration of the CRB into 3 fixed concentrations of TDB. FIG. 8E shows the percentage of target cell killing (normalized) for titration of TDB into 4 concentrations of fixed CRB. FIG. 8F shows correlation between the maximum percentage tumor cell killing activity in the Nuclight™ red assay compared to the maximum percentage activity in the caspase 3/7 assay (FIG. 8F; where filled, dark symbols show results using CD8+ T cells while unfilled, light symbols show results using pan T cells). FIG. 8G shows correlation between the maximum percentage activity in the Nuclight™ red assay compared to the maximum percentage activity in a Cell Titer Glo® assay (FIG. 8G; where dark symbols and light symbols show data for different tested TDBs).

Concentrations of factors such as IFNγ, IL2, and IL6 were also assessed. FIG. 9 shows changes in certain analyte concentrations (FIG. 9A— IFNγ; FIG. 9B—granzyme B; FIG. 9C— IL2; and FIG. 9D— IL6) in the supernatants from wells 6, 24, and 72 hours after addition of TDB. The individual curves in each graph represent data with different TDB clones.

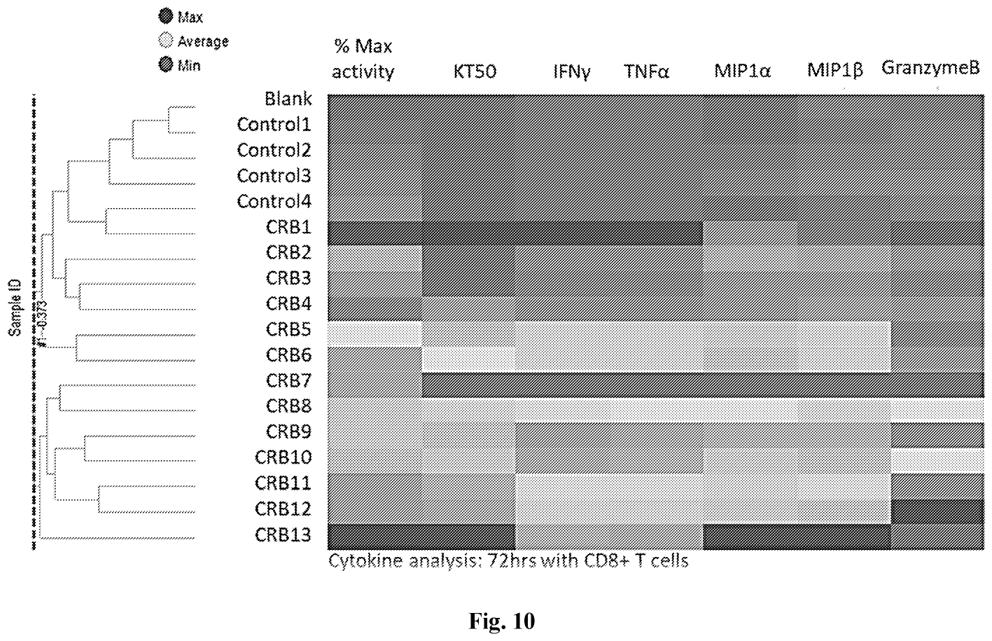

FIG. 10 shows a heat map ranking various CRB clones and controls based on multiple data readouts, for example KT50 of cell killing and changes in various cytokine concentrations. The cytokines were analyzed after 72 hours incubation with CD8+ T cells.

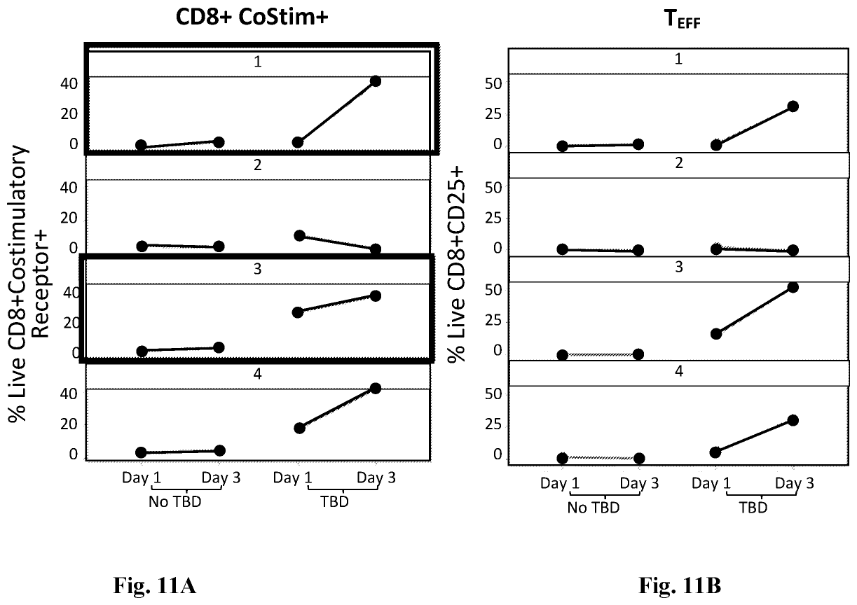

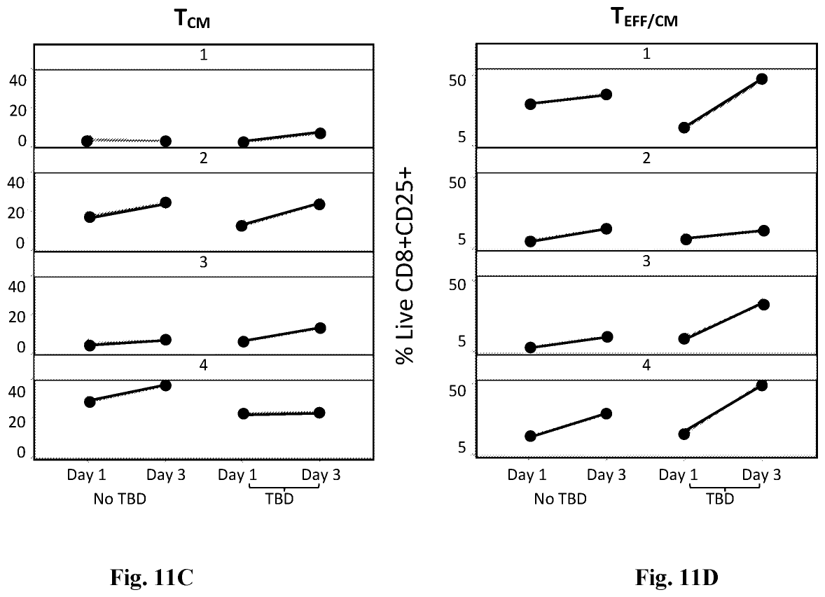

T cell subpopulations were assessed using immune cells from four different donors, 1, 2, 3, and 4. FIG. 11 shows increases in T cell subpopulations in immune cells from four donors over time after 1 or 3 days incubation with target cells, and with or without TDB. FIG. 11A CD8+ T cells; FIG. 11B T effector cells (Teff); FIG. 11C memory T cells (Tcm); and FIG. 11D ratio of effector to memory cells (Teff/Tcm).

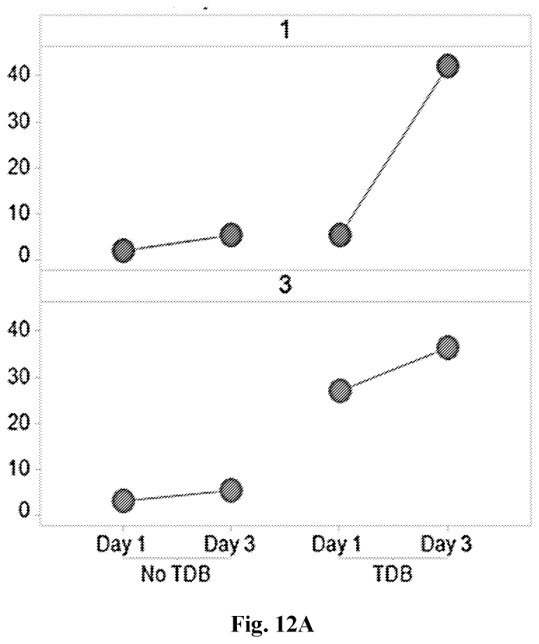

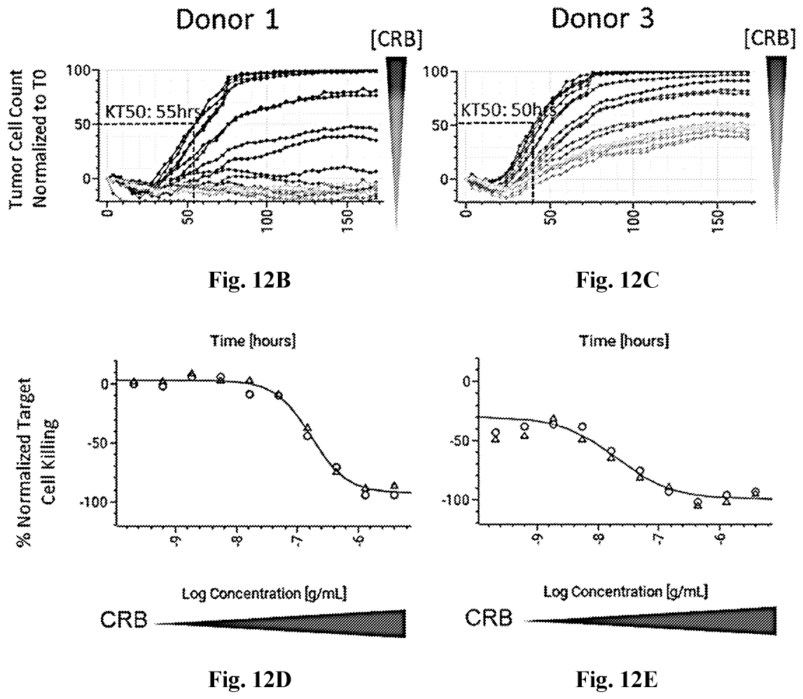

There were differences between donors 1 and 3 on CD8+ T cell proliferation with and without added TDB, as shown in FIG. 12A. FIG. 12B and FIG. 12C show comparisons of the rate of cell killing with increases in concentration of CRB for the two donors (1 and 3), and FIG. 12D and FIG. 12E show dose response curves corresponding to the data in FIGS. 12B and C.

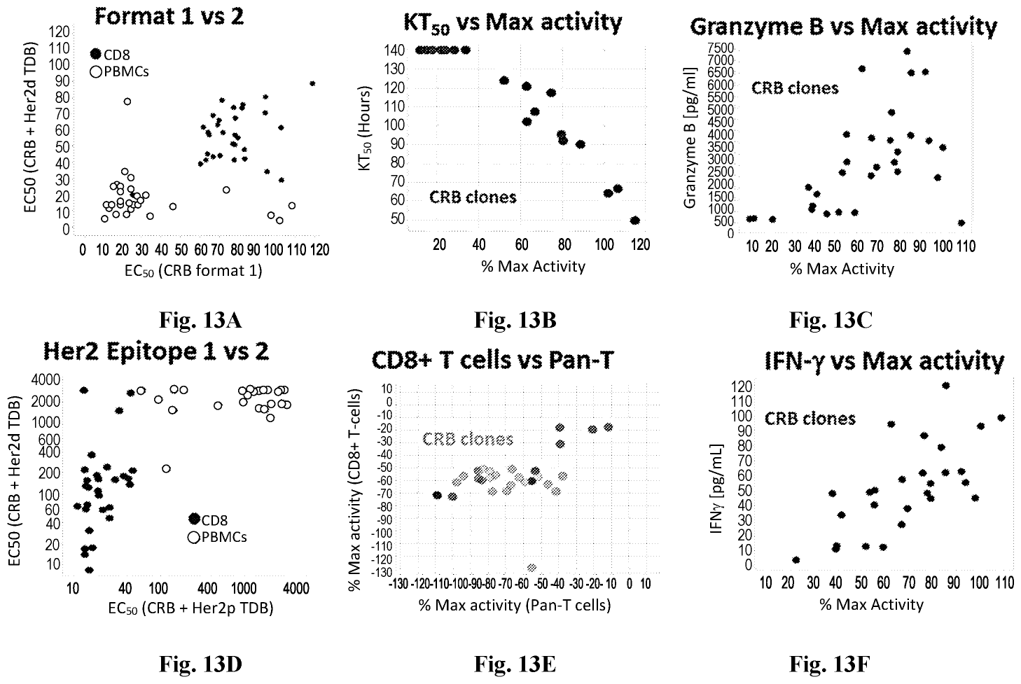

FIG. 13 shows correlations of multiple readouts. Specifically, FIG. 13A compares the EC50 of two different CRB molecules in the presence of target cells and either CD8+ T cells (filled, dark circles) or PBMCs (light, unfilled circles). FIG. 13B shows KT50 vs Max % activity of several different CRB clones in the presence of target cells and CD8+ T cells. FIG. 13C shows granzyme B vs Max % activity of various CRB clones with CD8+ T cells. FIG. 13D compares EC50 for the Her2d TDB and Her2p TDB (see FIG. 7) in the presence of CRB and with CD8+ T cells (filled circles) or PBMCs (unfilled circles). FIG. 13E shows comparison between max % activity of CD8+ T cells vs Pan T cells in the presence of several CRB clones. FIG. 13F compares IFNγ vs max activity of CD8+ T cells in the presence of several CRB clones.

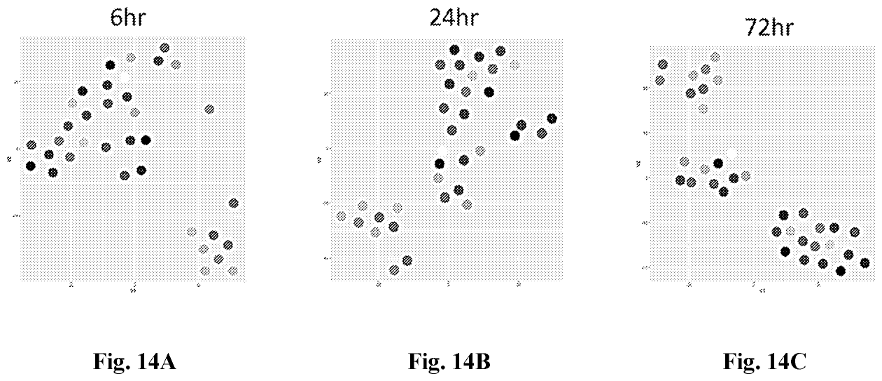

FIG. 14A-C shows a t-distributed stochastic neighbor embedding (t-SNE) machine learning algorithm cluster analysis of various TDB clones based on their killing and cytokine profiles over time (FIG. 14A 6 hr, FIG. 14B 24 hr, and FIG. 14C 72 hrs) to identify unique TDBs.

1. A method of assaying the activity of a potential immune cell engager, comprising:
(a) co-culturing target cells and immune cells in the presence of at least one potential immune cell engager, and
(b) assaying at least one of the following parameters, optionally wherein each parameter is assayed within the same co-cultured cell sample: (i) death of target cells, (ii) apoptosis of target cells, (iii) change in ATP concentration; and (iv) change in concentration of at least one analyte in supernatant from the co-cultured cells.

2. The method of claim 1, wherein the co-culturing comprises adding the target cells to wells of a multi-well cell plate, and adding immune cells and at least one potential immune cell engager to the target cells in the wells.

3. The method of claim 2, wherein the cell plate comprises 96 to 384 wells.

4. The method of claim 1, wherein the target cells are tumor cells or primary cells.

5. The method of claim 1, wherein the immune cells are T cells (such as CD8+ T cells, CD4+ T cells, CD3+ T cells, or Pan T cells), PBMC cells, or NK cells.

6. The method of claim 1, wherein the immune cells are derived from more than one donor.

7. The method of claim 1, wherein the target cells are transduced with a vector encoding a fluorescent nuclear protein that provides lower signal when cells are killed or undergo apoptosis, and wherein death of target cells is measured by loss of fluorescent nuclear protein signal.

8. The method of claim 1, wherein apoptosis of target cells is measured by increase of signal from a caspase 3/7-dependent fluorescent label.

9. The method of claim 1, wherein decrease in ATP concentration is measured by a luminescent label.

10. The method of claim 1, wherein supernatant from the co-culture is removed at least once after addition of the potential immune cell engager to the co-cultured cells, and wherein the concentration of at least one cytokine, chemokine, or T cell activity marker is measured, such as granzyme B, interferon gamma (IFNg), IL-10, IL-2, IL-6, IL-8, MIPla, MIP1b, or TNF-alpha (TNFa).

11. The method of claim 1, wherein the kinetics of at least one of parameters (i) to (iv) is determined.

12. The method of claim 1, wherein the time to 20%, 30%, 40%, 50%, 60%, 70%, 80%, 90%, or 100% target cell death is determined.

13. The method of claim 1, wherein a portion of the co-culture is removed to perform a flow cytometry analysis to determine presence of at least one immune cell marker, such as CD3, CD8, or CD4.

14. The method of claim 1, wherein the potential immune cell engager is a bispecific molecule.

15. The method of claim 1, wherein the potential immune cell engager is an antibody, such as a bispecific antibody.

16. The method of claim 15, wherein the antibody is a T cell dependent bispecific antibody (TDB).

17. The method of claim 1, wherein the method further comprises adding a costimulatory receptor bispecific antibody (CRB) to the co-cultured cells.

18. The method of claim 1, wherein at least two of parameters (i) to (iv) are determined, wherein the parameters are determined from the same co-culture sample or from the same well of a cell plate.
